(19) United States
(12) Patent Application Publication
(10) Pub. No.: US 2008/0226739 A1
(43) Pub. Date: Sep. 18, 2008

(54) Hierarchically self-assembling linear-dendritic hybrid polymers for delivery of biologically active agents

(76) Inventors: Kris C. Wood, Cambridge, MA (US); Paula Hammond Cunningham, Newton, MA (US); Steven Little, Allison Park, MA (US); Robert Langer, Newton, MA (US)

Correspondence Address: CHOATE, HALL & STEWART LLP, TWO INTERNATIONAL PLACE, BOSTON, MA, 02110, US

(21) Appl. No.: 11/473,806

(22) Filed: Jun. 22, 2006

Related U.S. Application Data
(60) Provisional application No. 60/692,916, filed on Jun. 22, 2005.
(60) Provisional application No. 60/710,572, filed on Aug. 23, 2005.

(51) Int. Cl. A61K 9/14; A61K 47/34; A61K 31/715; A61K 38/02; A61K 31/7052

(52) U.S. Cl. 424/497; 514/772.1; 514/54; 514/2; 514/44

(57) ABSTRACT

A linear-dendritic hybrid polymer for encapsulating biologically active materials. The hybrid polymer includes a ligand for a predetermined target, a dendron, and a polyethylene glycol (PEG) chain linking the ligand to the dendron.

This application claims priority from U.S. Provisional Applications Nos. 60/692,916, filed Jun. 22, 2005, and 60/710,572, filed Aug. 23, 2005, the contents of both of which are incorporated herein by reference.

This invention was supported in part by the Division of Materials Research of the National Science Foundation (DMR 9903380), the National Institutes of Health (EB00244), and the Office of Naval Research. The U.S. Government may have certain rights in the invention.

### FIELD OF THE INVENTION

This invention relates to delivery vehicles for biologically active agents such as polynucleotides, and, more specifically, to the use of linear-dendritic hybrid polymers in such delivery vehicles.

### BACKGROUND OF THE INVENTION

The application of nucleotide-based therapeutics in clinical medicine has the potential to revolutionize the treatment of human disease. To fully realize the potential for new medical advances in the post-genomic era, it is desirable to have safe and efficient delivery systems for nucleotide-based drugs.¹ Ideally, such systems will be nontoxic, non-immunogenic, and made from building blocks that are versatile to allow for optimal delivery to a range of cells or tissues of interest. The success of gene therapy is dependent upon the ability to deliver genes that express key proteins when and where they are needed. As of yet, no such therapies have been approved for clinical use, primarily because of the lack of versatile, safe, and efficient gene delivery systems. A suite of electrical, mechanical, and modified viral delivery systems have been investigated with some success, but these systems suffer from significant drawbacks.^{4,6} Notably, modified viruses often elicit severe immunogenicity, are prone to insertional mutagenesis, and are refractory to repeated administrations. Chemical delivery systems such as cationic linear polymers, dendrimers, or lipid-based reagents, while generally safer than their viral counterparts, typically lack the high efficiency or multiple functionalities required for in vivo administration. Moreover, even subtle synthetic modifications to these systems can dramatically influence existing biological properties (Luo, et al., Macromolecules, (2002), 35:3456). One of the most promising delivery approaches involves the use of cationic polymers, and a range of linear, branched, and dendritic polymers have been explored, including poly(β-amino esters), poly(ethylenimines), and poly(amidoamines), respectively. Unlike viral delivery systems, which are often highly immunogenic, prone to insertional mutagenesis, and refractory to repeated administrations, non-viral (polymeric) delivery systems can be synthesized with low immunogenicity and toxicity, though they frequently suffer from cytotoxicity, poor tissue targeting, rapid clearance from circulation, and low expression efficiency.¹

### DEFINITIONS

The term alkyl as used herein refers to saturated, straight- or branched-chain hydrocarbon radicals derived from a hydrocarbon moiety containing between one and twenty carbon atoms by removal of a single hydrogen atom. Examples of alkyl radicals include, but are not limited to, methyl, ethyl, propyl, isopropyl, n-butyl, tert-butyl, n-pentyl, neopentyl, n-hexyl, n-heptyl, n-octyl, n-decyl, n-undecyl, and dodecyl.

The term aryl as used herein refers to carbocyclic ring system having at least one aromatic ring including, but not limited to, phenyl, naphthyl, tetrahydronaphthyl, indanyl, indenyl, and the like. Aryl groups can be unsubstituted or substituted with substituents selected from the group consisting of branched and unbranched alkyl, alkenyl, alkynyl, haloalkyl, alkoxy, thioalkoxy, amino, alkylamino, dialkylamino, trialkylamino, acylamino, cyano, hydroxy, halo, mercapto, nitro, carboxyaldehyde, carboxy, alkoxycarbonyl, and carboxamide. In addition, substituted aryl groups include tetrafluorophenyl and pentafluorophenyl.

The term hydrocarbon, as used herein, refers to any chemical group comprising hydrogen and carbon. The hydrocarbon may be substituted or unsubstituted. The hydrocarbon may be unsaturated, saturated, branched, unbranched, cyclic, polycyclic, or heterocyclic. Illustrative hydrocarbons include, for example, methyl, ethyl, n-propyl, iso-propyl, cyclopropyl, allyl, vinyl, n-butyl, tert-butyl, ethynyl, cyclohexyl, methoxy, diethylamino, and the like. As would be known to one skilled in this art, all valencies must be satisfied in making any substitutions.

The terms substituted, whether preceded by the term “optionally” or not, and substituent, as used herein, refer to the ability, as appreciated by one skilled in this art, to change one functional group for another functional group provided that the valency of all atoms is maintained. When more than one position in any given structure may be substituted with more than one substituent selected from a specified group, the substituent may be either the same or different at every position. The substituents may also be further substituted (e.g., an aryl group substituent may have another substituent off it, such as another aryl group, which is further substituted with fluorine at one or more positions).

“Biomolecules”: The term “biomolecules”, as used herein, refers to molecules (e.g., proteins, amino acids, peptides, polynucleotides, nucleotides, carbohydrates, sugars, lipids, nucleoproteins, glycoproteins, lipoproteins, steroids, etc.) whether naturally-occurring or artificially created (e.g., by synthetic or recombinant methods) that are commonly found in cells and tissues. Specific classes of biomolecules include, but are not limited to, enzymes, receptors, neurotransmitters, hormones, cytokines, cell response modifiers such as growth factors and chemotactic factors, antibodies, vaccines, haptens, toxins, interferons, ribozymes, anti-sense agents, plasmids, DNA, and RNA.

“Polynucleotide”, “nucleic acid”, or “oligonucleotide”: The terms “polynucleotide”, “nucleic acid”, or “oligonucleotide” refer to a polymer of nucleotides. The terms “polynucleotide”, “nucleic acid”, and “oligonucleotide”, may be used interchangeably. Typically, a polynucleotide comprises at least three nucleotides. DNAs and RNAs are polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages). The polymer may also be a short strand of nucleic acids such as siRNA.

The terms “polynucleotide”, “nucleic acid”, or “oligonucleotide”, may also encompass nucleic acid based drugs, such as DNA, RNA, modified DNA, modified RNA, antisense oligonucleotides, expression plasmid systems, nucleotides, modified nucleotides, nucleosides, modified nucleosides, nucleic acid ligands (e.g. aptamers), intact genes, a promotor complementary region, a repressor complementary region, an enhancer complementary region, and combinations thereof. A promotor complementary region, a repressor complementary region, and/or an enhancer complementary region may be fully complementary or partially complementary to the DNA promotor region, repressor region, an enhancer region of a gene for which it is desirable to modulate expression.

“Polypeptide”, “peptide”, or “protein”: As used herein, a “polypeptide”, “peptide”, or “protein” includes a string of at least two amino acids linked together by peptide bonds. The terms “polypeptide, “peptide”, and “protein”, may be used interchangeably. Peptide may refer to an individual peptide or a collection of peptides. In some embodiments, peptides may contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a peptide may be modified, for example, by the addition of a chemical entity such as a carbohydrate group, a phosphate group, a farnesyl group, an isofarnesyl group, a fatty acid group, a linker for conjugation, functionalization, or other modification, etc. In one embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

The terms “polysaccharide” or “oligosaccharide”, as used herein, refer to any polymer or oligomer of carbohydrate residues. The polymer or oligomer may consist of anywhere from two to hundreds to thousands of sugar units or more. “Oligosaccharide” generally refers to a relatively low molecular weight polymer, while “starch” typically refers to a higher molecular weight polymer. Polysaccharides may be purified from natural sources such as plants or may be synthesized de novo in the laboratory. Polysaccharides isolated from natural sources may be modified chemically to change their chemical or physical properties (e.g., phosphorylated, cross-linked). Carbohydrate polymers or oligomers may include natural sugars (e.g., glucose, fructose, galactose, mannose, arabinose, ribose, and xylose) and/or modified sugars (e.g., 2′-fluororibose, 2′-deoxyribose, and hexose). Polysaccharides may also be either straight or branch-chained. They may contain both natural and/or unnatural carbohydrate residues. The linkage between the residues may be the typical ether linkage found in nature or may be a linkage only available to synthetic chemists. Examples of polysaccharides include cellulose, maltin, maltose, starch, modified starch, dextran, and fructose. Glycosaminoglycans are also considered polysaccharides. Sugar alcohol, as used herein, refers to any polyol such as sorbitol, mannitol, xylitol, galactitol, erythritol, inositol, ribitol, dulcitol, adonitol, arabitol, dithioerythritol, dithiothreitol, glycerol, isomalt, and hydrogenated starch hydrolysates.

“Small molecule”: As used herein, the term “small molecule” is used to refer to molecules, whether naturally-occurring or artificially created (e.g., via chemical synthesis), that have a relatively low molecular weight. Typically, small molecules are monomeric and have a molecular weight of less than about 1500 g/mol. Preferred small molecules are biologically active in that they produce a local or systemic effect in animals, preferably mammals, more preferably humans. In certain preferred embodiments, the small molecule is a drug. Preferably, though not necessarily, the drug is one that has already been deemed safe and effective for use by the appropriate governmental agency or body. For example, drugs for human use listed by the FDA under 21 C.F.R. §§ 330.5, 331 through 361, and 440 through 460; drugs for veterinary use listed by the FDA under 21 C.F.R. §§ 500 through 589, incorporated herein by reference, are all considered acceptable for use in accordance with the present invention.

“Bioactive agents”: As used herein, “bioactive agents” is used to refer to compounds or entities that alter, inhibit, activate, or otherwise affect biological or chemical events. For example, bioactive agents may include, but are not limited to, anti-AIDS substances, anti-cancer substances, antibiotics, immunosuppressants, anti-viral substances, enzyme inhibitors, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA or protein synthesis, anti-hypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics, and imaging agents. In certain embodiments, the bioactive agent is a drug.

A more complete listing of bioactive agents and specific drugs suitable for use in the present invention may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, Edited by Susan Budavari et al., CRC Press, 1996, and the United States Pharmacopeia-25/National Formulary-20, published by the United States Pharmcopeial Convention, Inc., Rockville Md., 2001, all of which are incorporated herein by reference.

### SUMMARY OF THE INVENTION

In one aspect, the invention is a linear-dendritic hybrid polymer for encapsulating biologically active materials. The hybrid polymer includes a ligand for a predetermined target, a dendron, and a polyethylene glycol (PEG) chain linking the ligand to the dendron. The PEG chain may be branched, and the hybrid polymer may include more than one ligand linked to the dendron by the PEG chain. The predetermined target may be multivalent. The PEG chain may include at least nine repeat units, for example, at least one hundred, at least five hundred, at least one thousand, at least five thousand, or at least ten thousand. The dendron may be a G3-G10 dendron, for example a G4-G6 dendron.

The dendron may include poly(amidoamine), polylysine, or polypropylenimine. The dendron may have a peptide based dendromer composition, a nucleic acid based composition, or a degradable cationic dendromer composition. The dendron may include functional groups having a pKa between about 5.5 and about 6.5. The ligand may include a nucleic acid ligand (e.g., aptamer), oligonucleotide, oligopeptide, polysaccharide, low-density lipoprotein (LDLs), folate, transferrin, asialycoprotein, gp120 envelope protein of the human immunodeficiency virus (HIV), enzymatic receptor ligand, sialic acid, glycoprotein, lipid, small molecule, bioactive agent, biomolecule, immunoreactive fragments such as the Fab, Fab′, or F(ab′)₂ fragments, protein, lipid, small molecule, bioactive agent, biomolecule, antibody, or antibody fragment. The ligand may be retained on the PEG chain through covalent or non-covalent interactions. The non-covalent interactions may be host-guest interactions, hydrogen bonding, metal coordination, hydrophobic interactions, or the interaction between biotin and avidin or streptavidin. The free ends of the dendron may include one or more of biotin, streptavidin, avidin, nitrile, amide, ester, thiol, halogen, tosylate, hydroxyl, alkyl, aryl, and alkylaryl.

In another aspect, the invention is a nanoparticle for use in encapsulating a biologically active agent including a quantity of the biologically active agent surrounded by a shell including the hybrid polymer. At least a portion of the biologically active agent may interact with free ends of the dendron via a non-covalent interaction. The non-covalent interaction may be a host-guest interaction, hydrogen bonding, metal coordination, hydrophobic interaction, pi-bonding, charge interaction, or the interaction between biotin and avidin or streptavidin. The nanoparticle may be between 25 nm and 2 microns in diameter, for example between 25 nm and 100 nm, between 100 nm and 500 nm, between 500 nm and 1 micron, or between 1 and 2 microns. The biologically active agent may be a polynucleotide, a small molecule, a bioactive agent, a polypeptide, a growth factor, or a glycosaminoglycan.

In another aspect, the invention is a composition for delivering the biologically active agent to patient including a plurality of nanoparticles. The composition may further include a carrier and may be suitable for administration by injection, as a suppository, orally, as an inhalant, or topically.

In another aspect the invention is a method of producing a ligand-functionalized polyethylene glycol-dendromer hybrid polymer. The method includes attaching a predetermined ligand to a free end of a PEG chain using a covalent or non-covalent traction and using a second free end of the PEG chain as the core of a dendron. Using a second free end may include alternately reacting a primary amine in chemical communication with the PEG chain with methyl acrylate and ethylene diamine. The PEG chain may have more than two free ends and attaching may include attaching a ligand to all but one of the free ends. Using a second free end may include synthesizing a G3-G10 dendron, for example, a G4-G6 dendron. The method may further include modifying at least a portion of free ends of the dendron.

In another aspect, the invention is a method of encapsulating a biologically active material. The method includes providing a hybrid polymer including a ligand for a predetermined target, a dendron, and a PEG chain linking the ligand to the dendron, and incubating the hybrid polymer with the biologically active material under conditions where the hybrid polymer forms vesicles surrounding a quantity of the biologically active material. At least a portion of the vesicle may be between 25 nm and 2 microns in diameter, for example, between 25 nm and 100 nm, between 100 nm and 500 nm, between 500 nm and 1 micron, or between 1 and 2 microns.

### BRIEF DESCRIPTION OF THE DRAWING

The invention is described with reference to the several figures of the drawing, in which,

FIG. 1 is a schematic depicting self-assembly of ligand-functionalized linear-dendritic diblock copolymers according to an embodiment of the invention with plasmid DNA.

FIG. 2A is a schematic illustrating the molecular structure-function relationship in mannose-PEG-PAMAM G3.0.

FIG. 2B is a schematic showing the structure of an exemplary linear-dendritic polyplex showing relative positions of functional elements (not to scale).

FIG. 3 is a schematic depicting intracellular barriers to receptor-mediated gene delivery.

FIG. 4A is a schematic illustrating the synthesis of linear-dendritic hybrid polymers according to an embodiment of the invention.

FIG. 4B is a ¹H NMR spectrum of mannose-PEG-PAMAM-G4.0 with assigned structural peaks (A: δ_{SUGAR} (CH2OH)=3.88; B: δ_{PEG} (CH2CH2O)=3.65; C: δ_{PAMAM} (CH2CONHCH2)=3.27; D: δ_{PAMAM} (next to 1°, 3° amines)=2.45-3.1; δ_{PAMAM} (CH2CONH)=2.37)

FIG. 5 includes infrared spectra demonstrating exponential dendron growth in sugar-PEG-PAMAM (a) G0.0, (b) G2.0, and (c) G4.0 systems. Growth of amide (˜3000-3500 cm⁻¹) and carbonyl (˜1400-1800 cm⁻¹) peaks are highlighted.

FIG. 6A is a series of photographs of 1% agarose electrophoresis gels demonstrating DNA binding at indicated mass ratios.

FIG. 6B is a graph illustrating the diameters, as measured via dynamic light scattering (DLS), of particles produced according to various embodiments of the invention.

FIG. 6C is a pair of transmission electron micrographs of particles produced according to an embodiment of the invention.

FIGS. 7A and B are graphs illustrating the level of transfection of P388D1 macrophages bearing the mannose receptor. (A) Transfection by linear-dendritic polyplexes with and without the mannose ligand and in the presence of 0.1 mg/well soluble mannose. * indicates p<0.04; ** indicates p<0.002 (Two-tailed, unpaired Student's T-Test). Results normalized to an optimized formulation of PEI (2:1 PEI:DNA, serum-free, no free mannose added). (B) Serum stability is demonstrated via transfection in the presence of serum proteins. Results normalized to an optimized formulation of PEI (2:1 PEI:DNA, 10% serum, no free mannose added). All results are given as average+/−standard error.

FIGS. 8A and B are graphs illustrating this level of transfection of HepG2 hepatocytes bearing the asialoglycoprotein receptor. (A) Transfection by linear-dendritic polyplexes with and without the galactose ligand * indicates p<0.06; ** indicates p<0.03 (Two-tailed, unpaired Student's T-Test). Results normalized to PEI (serum free, no free galactose added)=1.0. (B) Serum stability is demonstrated via transfection in the presence of serum proteins. Results normalized to PEI (10% serum, no free mannose added)=1.0. All results are given as average+/−standard error.

FIG. 9 is a series of graphs illustrating the relative viability of (A) P388D1 macrophages and (B) HepG2 hepatocytes 72 h following transfection at indicated polymer/DNA mass ratios (control cells untreated). All results are given as average +/− standard error.

FIG. 10 is a schematic diagram of a two-step aqueous synthesis of hybrid polymers according to an embodiment of the invention.

FIG. 11 is a graph illustrating the transfection of DU145 human prostate cancer cells by peptide-modified hybrid polymers according to an embodiment of the invention.

FIG. 12 is a graph illustrating antibody levels in mice treated with linear-dendritic hybrid complexes employing mannose to selectively deliver a polynucleotide encoding beta-galactosidase.

### DETAILED DESCRIPTION OF CERTAIN PREFERRED EMBODIMENTS

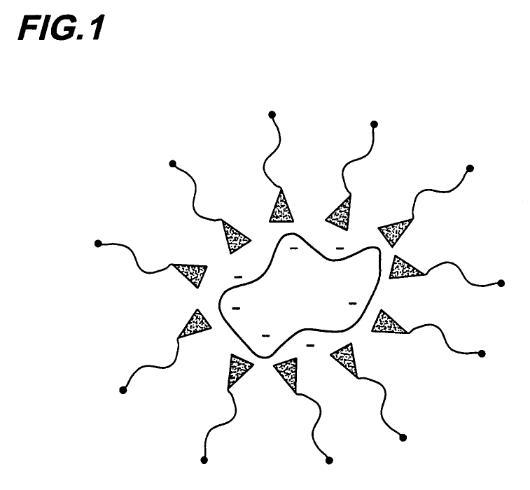

Herein, we present a new family of multifunctional gene delivery polymers with a wide array of properties (i.e., blood stability, cellular targeting, DNA binding, and endosomal buffering capacity) that can be independently tuned in a modular fashion to address each of the barriers to effective gene delivery. In one embodiment, the invention includes the design, synthesis, and testing of a unique family of hierarchically structured linear-dendritic hybrid polymers (FIG. 1) that self-assemble with polynucleotides and other biologically active materials to form stable nanoparticles with a series of concentric, functional “shells” possessing independently-tunable properties necessary for effective targeted delivery. The resultant ligand-functionalized systems demonstrate receptor-mediated delivery to targeted cells with robust serum stability, transfection efficiencies exceeding the most efficient commercially available polymer, poly(ethylenimine) (PEI), and low toxicity at concentrations one to two orders of magnitude higher than those at which PEI is toxic. This is in direct contrast to traditional polymeric gene delivery systems, which condense DNA into globular nanoparticles that lack well-defined hierarchical organization and do not have the facility for fine-tuning of functional parameters independently. Targeting and expression levels may be modulated independently by the choice of ligand and dendrimer species, respectively. Further, these systems represent a platform on which additional functionalities may be added to impart properties such as vector unpackaging and nuclear targeting.^{8,9}

In an exemplary embodiment, the system includes linear poly(ethylene glycol) (PEG) and dendritic poly(amidoamine) (PAMAM). These linear-dendritic diblock copolymers self-assemble with DNA to yield nanoparticles (150 nm) with a core of primary amines used to neutralize and condense negatively charged DNA, an inner shell of secondary and tertiary amines to promote efficient intracellular delivery via endosomal escape, and an outer shell of PEG, a highly hydrophilic, flexible polymer to sterically stabilize the nanoparticles, decrease nonspecific uptake, and dramatically improve serum stability and circulation half-life in vivo by preventing opsonization by plasma proteins. Finally, these diblock copolymer systems can be functionalized with targeting ligands to be displayed on the outer surface of the particle to promote receptor-mediated uptake by target cells (See FIG. 1). In vitro studies, discussed below, with PEG-PAMAM copolymers functionalized with the small molecule ligands mannose (for CD206 receptor-positive macrophage cells) and galactose (for asialoglycoprotein receptor-positive hepatocytes), indicate that these systems can transfect cells at levels nearly equal to that of branched poly(ethylenimine) (PEI, one of the best known in vitro transfection reagents) in the absence of serum proteins, and better than 15-fold more efficiently in the presence of serum proteins. Moreover, these systems exhibit selective targeting to cells bearing the receptor of interest and exhibit no measurable toxicity at concentrations 100-fold higher than those at which PEI is toxic.

### Dendrimers

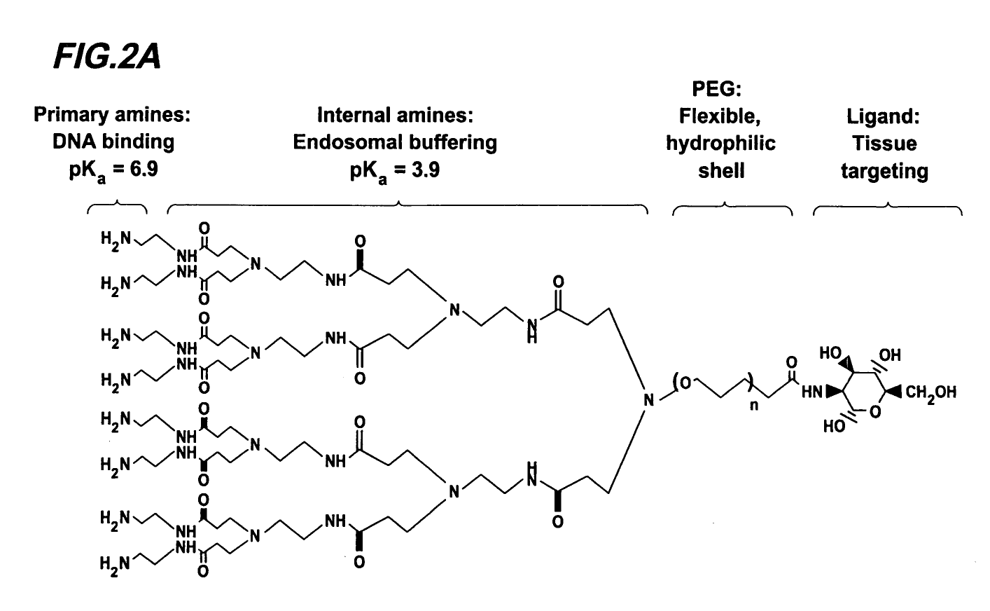

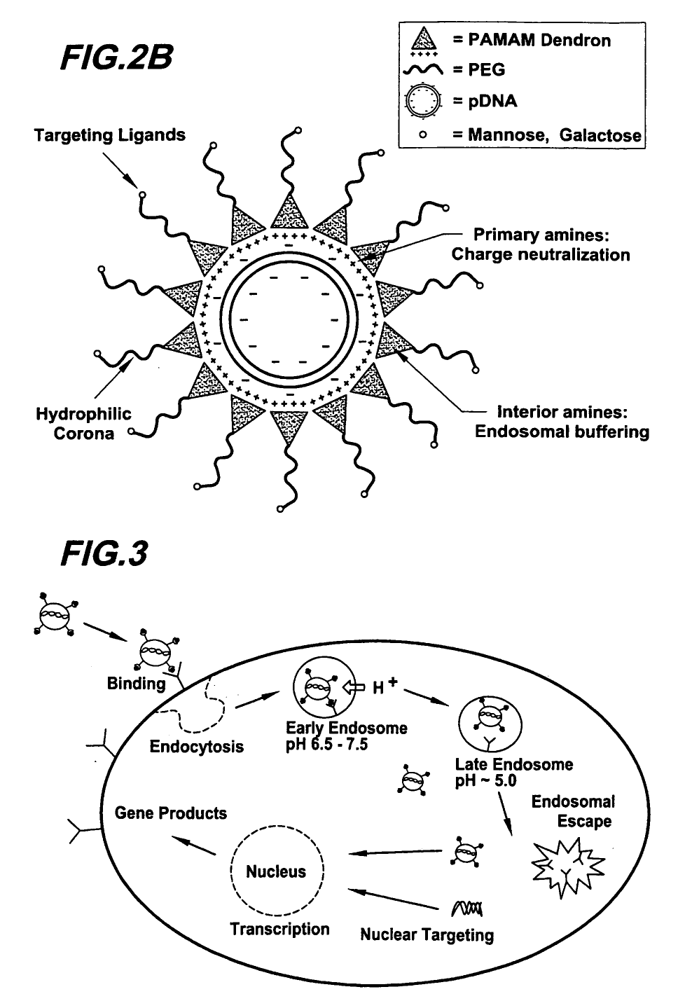

Linear-dendritic hybrid polymers 10 were designed based on the hypothesis that these unique polymer architectures, which possess functionalities that are both chemically orthogonal and physically separated, could self-assemble with polynucleotides and other biologically active materials to yield nanoparticles with an outer shell of targeting ligands 12 accessible to cell surface receptors, a hydrophilic corona of flexible chains 14 designed to prevent protein opsonization, plasma clearance, and non-specific uptake, and an interior of amine groups on dendrons 16 to promote DNA binding and escape from endosomal vesicles into the cytoplasm. (See FIGS. 2 and 3).¹⁰

The dendron, a non-centrosymmetric dendritic polymer produced from a monofunctional core, serves two functions in the hybrid polymer. The free ends of the dendron 18 (FIG. 2) interact favorably (e.g., the interaction is energetically favorable) with the material being encapsulated in the nanoparticle, allowing the hybrid polymers 10 to surround or encapsulate the material in a vesicle. The internal portions of the dendron 20 act as a buffer. As the vesicle approaches a cell 22 (FIG. 3), the ligands 12 interact with receptors on cell 22, causing the cell to endocytose the vesicle, which passes into the cell 22 in early endosome 24. The interior of this endosome has a pH of about 6.5-7.5. The cell begins to pump protons into the endosome 24 to form late endosome 26, which has a pH of about 5. Without the dendron, at this point, the late endosome would merge with one or more other endosomes to form a lysosome. The pH would continue to decrease, and the contents of the lysosome would be digested into their constituent parts, which would then be used as building blocks for cellular synthetic processes. Any DNA delivered to the cell would not make it to the nucleus intact. Without being limited by any particular theory, it is believed that the buffering action of the internal portions 20 of the dendron causes the cell to pump many more protons into the endosome to reduce the pH. Counterions such as chlorine must accompany the protons to maintain charge balance. Eventually, the volume of these ions and any water solvating them becomes too great, and the endosome bursts (endosomal escape), releasing the contents of the vesicle into the cytoplasm and, for example, giving any polynucleotides delivered by the dendrimeric vesicles an opportunity to reach the nucleus.

Dendrimer Compositions

In one embodiment, the dendrimer is a polyamidoamine dendrimer. Dendrons may be synthesized from monofunctional cores. One skilled in the art will recognize that other dendrimer compositions, such as polylysine, poly(propylenimine), peptide and DNA based dendrimers, and degradable cationic dendrimers, may also be employed. Indeed, any aminated or polyelectrolyte dendrimer having internal buffering groups with a pKa of about 5.5 to about 7.5 will provide the buffering capacity to enable endosomal escape. Exemplary dendrimer compositions and synthetic methods may be found in U.S. Pat. Nos. 6,113,946, 4,631,337, 4,558,120, 4,871,779, 4,857,599, and 5,648,186, Sadler, et al., “Peptide dendrimers: applications and synthesis,” Reviews in Molecular Biotechnology, 90:195-229 (2002), Stiriba, et al., “Dendritic Polymers in Biomedical Applications: From Potential to Clinical Use in Diagnostics and Therapy,” Angewante Chemie International Edition, 41:1329-1334 (2002), and Funhoff, et al., “Polymer Side-Chain Degradation as a Tool to Control the Destabilization of Polyplexes,” Pharmaceutical Research, 21:170-176 (2004), the contents of all of which are incorporated herein by reference. One skilled in the art will recognize how to adapt the methods of these publications to make dendrons by using monofunctional cores.

The dendron size is determined by the number of synthetic generations. For each generation, the volume of the dendron increases faster that its surface area, so that the ultimate possible size of the dendron is determined by steric hindrance at the free ends where the next generation of monomer is added. The desired size of the dendron depends on the desired buffering capacity, any toxicity to the cell that the hybrid polymer may exhibit, and the desired reaction time, since each generation must be added sequentially. Increased dendron size increases transfection efficiency but also increases possible toxicity, since the PEG chains may not be able to mask the charge of the toxic cationic groups if the dendron is too large. Longer PEG chains may be used with larger dendrons to reduce their toxicity. The dendrons may be prepared with between 3 and 10 generations, for example, 4-6 generations. Hybrid polymers with G6 dendrons allow transfection efficiencies greater than that of poly(ethylenimine) encapsulated material.

Modification of Primary Amine

The free ends of dendrimers formed with aminated monomers are primary or secondary amines. Such groups are appropriate for forming vesicles containing negatively charged materials such as polynucleotides. However, the free ends may be modified using standard organic chemistry techniques so that they are negatively charged or capable of engaging in other types of interactions with biologically active materials. In addition to charge interactions, exemplary non-covalent interactions include host-guest interactions, metal coordination, hydrophobic interactions, and hydrogen bonding interactions, all of which are described below. Exemplary methods of terminating dendrimers with groups such as nitrites, amides, esters, thiols, halogens, tosylates, hydroxyl, and other groups are disclosed in U.S. Pat. Nos. 4,507,466 and 4,713,975, the entire contents of both of which are incorporated herein by reference. Alternatively or in addition, the free ends of the dendrons may be biotinylated, as described in U.S. Patent Publication No. 20030096280, the entire contents of which are incorporated herein. In this embodiment, the material that is being encapsulated by the hybrid polymer may be derivitized with avidin or streptavidin. In another example, the free ends may be transformed to phenyl or other aryl groups or various hydrophobic groups (hydrocarbons, including alkyl groups) to enable the hybrid polymers to interact with biologically active materials via pi-bonding or van der Waals forces. Where the end groups are converted to hydrophobic moieties, the hybrid polymers may behave as the shell of a micelle, providing a barrier between a hydrophobic material and an aqueous environment. As described below, the interaction may be enhanced by functionalizing biologically active groups with complementary materials (e.g., hydrogen bond receptors or donors).

Where larger chemical groups, for example, a PEG chain, are used to convert the primary amines of the dendrimer, they may hinder one another or may not be able to convert every primary amine site, e.g., of a dendron. In this example, the remaining primary amine sites will continue to contribute to the buffering capacity of the dendron or may be converted to a smaller chemical group that will not interfere in the interaction between the hybrid polymer and the biologically active material. In some embodiments, at least 10%, at least 20%, at least 50%, at least 75%, or at least 90% of the free ends of the dendron are converted to another group.

Agents Delivered by Linear Dendritic Hybrid Polymers

A variety of biologically active material may be encapsulated by linear dendritic hybrid polymers produced according to an embodiment of the invention. Exemplary biologically active materials include biomolecules, small molecules, and bioactive agents as defined elsewhere herein. For example, the biologically active material may be a growth factor. Exemplary growth factors include but are not limited to VEGF or another growth factor, e.g., activin-A (ACT), retinoic acid (RA), epidermal growth factor, bone morphogenetic protein, TGF-β, hepatocyte growth factor, platelet-derived growth factor, TGF-α, IGF-I, IGF-II, hematopoietic growth factors, heparin binding growth factor, peptide growth factors, erythropoietin, interleukins, tumor necrosis factors, interferons, colony stimulating factors, acidic and basic fibroblast growth factors, nerve growth factor (NGF), or muscle morphogenic factor. Exemplary materials that may be encapsulated by hybrid polymers include charged materials such as heparin sulfate and glycosaminoglycans.

In another embodiment, polynucleotides are encapsulated by the hybrid polymers. For example, DNA vaccines may be delivered to cells to induce expression of particular antibodies, promote the production of particular proteins, or stimulate certain cellular metabolic activities. For example, production of particular proteins can block the metabolic activity of tumor or other disease cells. In another embodiment, use of DNA to promote the production of an antibody may obviate administration of a disease-causing agent to a patient as either a live or dead culture, reducing the risk of disease from vaccine.

While negatively charged materials such as polynucleotides, heparin sulfate, and glycosaminoglycans may easily be delivered by hybrid polymers with unmodified free ends, it may be desirable to modify the free ends of the hybrid polymers to better enable them to encapsulate neutrally or positively charged materials, as described above.

### PEG Linker Chains

The PEG chains increase the circulation time of the hybrid polymer-walled vesicles, in part by increasing the resistance of the vesicles to proteases. In addition, the PEG chains render the hybrid polymer-walled vesicles resistant to opsonization and agglomeration in serum while reducing toxicity and in vivo immunological response, increasing circulation time by as much as two orders of magnitude with respect to branched PEI. Nine to 10 repeat units are sufficient to provide this benefit, and longer chains (e.g., 500 mers) increase that resistance in comparison to shorter chains. In some embodiments, even for very long PEG chains (e.g., 5000 mers) and very small targeting ligands (e.g., simple sugars), the PEG chain does not interfere with cellular recognition of the targeting chain. As a result, the PEG chains may be arbitrarily long, for example, 50 mers, 100 mers, 1000 mers, 5000 mers, or 10,000 mers long, or longer. The length of the PEG chain may be limited if excessively long chains prove to be toxic for particular cells.

The PEG linker chains may also be branched, so that more than one ligand may be tethered to a single dendron. Branched PEG is commercially available, for example, from Nektar Therapeutics (San Carlos, Calif.). PEG chains functionalized with a variety of different end groups are available from Sigma. Standard organic chemistry techniques may be used to attach PEG chains to dendrons and to desired ligands. The skilled artisan is referred to Hermanson, Bioconjugate Techniques, (Academic Press, San Diego, 1996), the contents of which are incorporated herein by reference, for exemplary chemistries.

### Ligands

Practically any ligand may be attached to the end of the PEG linker chain to serve as a targeting agent for a receptor on a desired cell. A variety of targeting agents that direct pharmaceutical compositions to particular cells are known in the art (see, for example, Cotton, et al., Methods Enzym. 217:618; 1993; incorporated herein by reference). The targeting agents may be included throughout the particle or may be only on the surface. The ligand may be a protein, peptide, carbohydrate, polysaccharide, glycoprotein, lipid, small molecule, bioactive agent, biomolecule, etc. A ligand may target any part or component of a tissue. For example, ligands may exhibit an affinity for an epitope or antigen on a tumor or other tissue cell, an integrin or other cell-attachment agent, an enzyme receptor, an extracellular matrix material, or a peptide sequence in a particular tissue. One skilled in the art will recognize that the target need not be healthy tissue or a tumor but a particular form of tissue damage or disease. The ligand may be used to target specific cells or tissues or may be used to promote endocytosis or phagocytosis of the particle. Examples of ligands include, but are not limited to, antibodies and antibody fragments, nucleic acid ligands (e.g., aptamers), oligonucleotides, oligopeptides, polysaccharides, low-density lipoproteins (LDLs), folate, transferrin, asialycoproteins, gp120 envelope protein of the human immunodeficiency virus (HIV), carbohydrates, polysaccharides, enzymatic receptor ligands, sialic acid, glycoprotein, lipid, small molecule, bioactive agent, biomolecule, immunoreactive fragments such as the Fab, Fab′, or F(ab′)₂ fragments, etc.

In one embodiment, peptides or peptide fragments may be attached to the PEG tether. Appropriate peptide sequences for particular cells may be selected using yeast or phage display methods such as those disclosed in Vidal, et al., Oncogene, (2004), 23:8859-8867; Arap, et al., Cancer Cell, (2004) 6:275-284; Chen, et al., Chem Biol, (2004), 11:1081-1091; Zurita, et al., Cancer Res., (2004) 64:435-439; Zurita, et al., J. Control Release, (2003) 91(183-6); Muller, et al., Nat. Biotechnol, (2003) 21:1040-1046; Yao, et al., Am. J. Pathol., (2005) 166:624-636; Marchio, et al., Blood, (2005) 105: 2802-2811; Romanov, Medicinal Chemistry Reviews—Online, (2005) 2:219-229, Begent, R. H. J. et al., Nature Medicine, (1996) 2: 979-984; Chester, K. A. et al., Lancet, (1994) 343: 455-456, the contents of all of which are incorporated herein by reference. Peptides may also be selected using the methods disclosed in Scott, et al., Science, (1990) 249:386-90, the contents of which are incorporated herein by reference. Alternatively or in addition, antibodies for particular cells or cell receptors may be selected in the same manner. Where a peptide is employed as a targeting agent, it may be desirable to employ an aqueous-based method to synthesize the hybrid polymers, for example, as described in Example 5.

Alternatively or in addition, the PEG linker may be functionalized with avidin or streptavidin. In this embodiment, a generic hybrid polymer may be modified to target a specific receptor by incubating it with a biotinylated targeting agent for the particular receptor. Alternatively, the PEG linker may be functionalized with biotin, and a targeting agent functionalized with streptavidin or avidin may be incubated with the hybrid polymer to link them through affinity interactions.

Other non-covalent interactions may also be employed to link targeting agents to hybrid polymers according to an embodiment of the invention. Exemplary non-covalent interactions include the following:

Metal Coordination: For example, a polyhistidine may be attached to the targeting agent, and a nitrilotriacetic acid can be attached to the PEG linker. A metal, such as Ni+2, will chelate the polyhistidine and the nitrilotriacetic acid, thereby binding the targeting agent to the hybrid polymer.

Hydrophobic interactions: For example, a hydrophobic tail, such as polymethacrylate or an alkyl group having at least about 10 carbons, may be attached to the targeting agent and the PEG chain. The hydrophobic tail on the targeting agent will adsorb onto the hydrophobic shell of a hybrid polymer encapsulated nanoparticle, or individual hybrid polymers may interact with the targeting agent before formation of the nanoparticle. Other hydrophobic oligomers, such as polyorthoester, polysebacic anhydride, or polycaprolactone, may also be employed to create a hydrophobic group on either the hybrid polymer or the targeting agent.

Host-Guest Interactions: For example, a macrocyclic host, such as cucurbituril or cyclodextrin, may be attached to the PEG linker and a guest group, such as an alkyl group, a polyethylene glycol, or a diaminoalkyl group, may be attached to the targeting agent; or conversely, the host group may be attached to the targeting agent and the guest group may be attached to the PEG linker. In one embodiment, the host and/or the guest molecule may be attached to the targeting agent via a linker, such as an alkylene linker or a polyether linker.

Hydrogen Bonding Interactions: For example, an oligonucleotide having a particular sequence may be attached to the PEG linker, and an essentially complementary sequence may be attached to the targeting agent such that it does not disrupt the binding affinity of the targeting agent for its target. The targeting agent will then bind to the hybrid polymer via complementary base pairing with the oligonucleotide attached to the controlled release polymer system. Two oligonucleotides are essentially complimentary if at least about 80% of the nucleic acid bases on one oligonucleotide form hydrogen bonds via an oligonucleotide base pairing system, such as Watson-Crick base pairing, reverse Watson-Crick base pairing, Hoogsten base pairing, etc., with a base on the second oligonucleotide. In some embodiments, it is desirable for an oligonucleotide sequence attached to the hybrid polymer to form at least about 6 complementary base pairs with a complementary oligonucleotide attached to the targeting agent.

### Delivery

The hybrid polymers described herein may be used to encapsulate a variety of materials. The vesicles or particles formed thereby may range in diameter from about 25 nm to 1 or 2 microns, for example, between 25 nm and 100 nm, between 100 nm and 500 nm, between 500 nm and 1 micron, or between 1 and 2 microns. We have found that higher concentration formulations of the hybrid polymer-shelled vesicles may be administered in comparison to PEI-encapsulated polynucleotides. In vitro, cells experience little or no toxicity when exposed to concentrations over double levels where PEI-encapsulated material exhibits significant toxicity.

Once the particles have been prepared, they may be combined with pharmaceutical acceptable carriers to form a pharmaceutical composition. While the composition may be injectable or administrable as a suppository, it is more convenient when the composition is orally administrable, either through ingestion or as an inhalant. To this end, the particles produced according to an embodiment of the invention may be sufficiently small to traverse the intestinal mucosa or the alveolar wall. The size of the particle may be optimized for stability and increased uptake. One skilled in the art will recognize that the optimum particle size may vary depending on the nature of the drug being delivered.

As used herein, the term “pharmaceutically acceptable carrier” means a non-toxic, inert solid, semi-solid or liquid filler, diluent, encapsulating material or formulation auxiliary of any type. Remington's Pharmaceutical Sciences Ed. by Gennaro, Mack Publishing, Easton, Pa., 1995, discloses various carriers used in formulating pharmaceutical compositions and known techniques for the preparation thereof. Some examples of materials which can serve as pharmaceutically acceptable carriers include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as TWEEN™ 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate. Coloring agents, releasing agents, coating agents, sweetening, flavoring and perfuming agents, preservatives and/or antioxidants can also be present in the composition, according to the judgment of the formulator.

The pharmaceutical compositions of the invention can be administered to a patient by any means known in the art including oral and parenteral routes. The term “patient”, as used herein, refers to humans as well as non-humans, including, for example, mammals, birds, reptiles, amphibians, and fish. Preferably, the non-humans are mammals (e.g., a rodent, a mouse, a rat, a rabbit, a monkey, a dog, a cat, a primate, or a pig). Non-edible compositions may be administered by injection (e.g., intravenous, subcutaneous or intramuscular, intraperitoneal injection), rectally, vaginally, topically (as by powders, creams, ointments, or drops), or by inhalation (as by sprays).

Powders and sprays can contain, in addition to hybrid polymer-encapsulated vesicles, excipients such as lactose, talc, silicic acid, aluminum hydroxide, calcium silicates, and polyamide powder, or mixtures thereof. Sprays can additionally contain customary propellants such as chlorofluorohydrocarbons.

Pharmaceutical compositions for oral administration can be liquid or solid. Liquid dosage forms suitable for oral administration of inventive particles include pharmaceutically acceptable emulsions, microemulsions, solutions, suspensions, syrups, and elixirs. In addition to an encapsulated or unencapsulated particle, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants, wetting agents, emulsifying and suspending agents, sweetening, flavoring, and perfuming agents. As used herein, the term “adjuvant” refers to any compound that is a nonspecific modulator of the immune response. In certain preferred embodiments, the adjuvant stimulates the immune response. Any adjuvant may be used in accordance with the present invention. A large number of adjuvant compounds is known in the art (Allison Dev. Biol. Stand. 92:3-11, 1998; Unkeless et al. Annu. Rev. Immunol. 6:251-281, 1998; and Phillips et al. Vaccine 10:151-158, 1992).

Solid dosage forms for oral administration include capsules, tablets, pills, powders, and granules. In such solid dosage forms, the encapsulated or unencapsulated particle is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art.

It will be appreciated that the exact dosage of the inventive particle is chosen by the individual physician in view of the patient to be treated. In general, dosage and administration are adjusted to provide an effective amount of the desired active agent to the patient being treated. As used herein, the “effective amount” of a substance refers to the amount necessary to elicit the desired biological response. As will be appreciated by those of ordinary skill in the art, the effective amount of encapsulated active agent may vary depending on such factors as the desired biological endpoint, the active agent to be delivered, the target tissue, the route of administration, etc. For example, the effective amount of inventive particles containing an anti-cancer drug might be the amount that results in a reduction in tumor size by a desired amount over a desired period of time. Additional factors which may be taken into account include the severity of the disease state; age, weight and gender of the patient being treated; diet, time and frequency of administration; drug combinations; reaction sensitivities; and tolerance/response to therapy.

The particles of the invention may be compounded with a carrier in dosage unit form for ease of administration and uniformity of dosage. The expression “dosage unit form” as used herein refers to a physically discrete unit of conjugate appropriate for the patient to be treated. It will be understood, however, that the total daily usage of the compositions according to an embodiment of the invention will be decided by the attending physician within the scope of sound medical judgment. For any particle composition, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. Therapeutic efficacy and toxicity of particle materials and the drugs delivered thereby can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices are preferred. The data obtained from cell culture assays and animal studies is used in formulating a range of dosage for human use.

### EXAMPLES

### Example 1

### Synthesis of a Hybrid Polymer

General Considerations Bifunctional Fmoc-PEG-NHS (Mₙ=3500) and HCl.NH₂-PEG-COOH (Mₙ=3400) were purchased from Nektar Therapeutics (Birmingham, Ala.) and used without further purification (both possessed substitution values>99%). Methyl acrylate (99+%) and ethylene diamine (99+%) were purchased from Sigma-Aldrich (St. Louis, Mo.) and distilled prior to use. D-mannosamine HCl, 1-amino-1-deoxy-b-D-galactose, and hyperbranched poly(ethylenimine) (PEI, Mₙ=25000) were purchased from Sigma-Aldrich (St. Louis, Mo.). Plasmid DNA containing the firefly luciferase reporter gene and CMV promoter sequence (pCMV-Luc) was purchased from Elim Biopharmaceuticals (San Francisco, Calif.) and used without further purification. HepG2 human hepatocellular carcinoma cells and P388D1 murine macrophages were purchased from American Type Culture Collection (Manassas, Va.) and grown at 37° C. in 5% CO₂. HepG2 cells were grown in 90% Dulbecco's modified Eagle's medium supplemented with 10% fetal bovine serum, 100 units/mL penicillin, and 100 μg/mL streptomycin. P388D1 macrophages were grown in 90% RPMI 1640 medium supplemented with 10% fetal bovine serum, 100 units/mL penicillin, 100 μg/mL streptomycin, 2.5 mg/mL D-glucose, 10 mM HEPES, and 1 mM sodium pyruvate. Bright-Glo® luciferase assay detection kits were purchased from Promega (Madison, Wis.) and used according to the manufacturer's specifications. All other materials and solvents were used as received without further purification.

Instrumentation: ¹H NMR spectra were recorded at room temperature using a Varian Mercury 300 MHz instrument. FTIR spectra of films cast on polished KBr pellets were recorded on a Nicolet Magna-IR 550 spectrometer. A ZetaPALS dynamic light scattering detector (Brookhaven Instruments, 15 mW laser, incident beam 676 nm) was used for particle sizing. Luminescence from reporter gene expression studies was measured using a Veritas Microplate Luminometer. Optical absorbance was measured using a SpectraMax 190 microplate reader (Molecular Devices, Sunnyvale, Calif.).

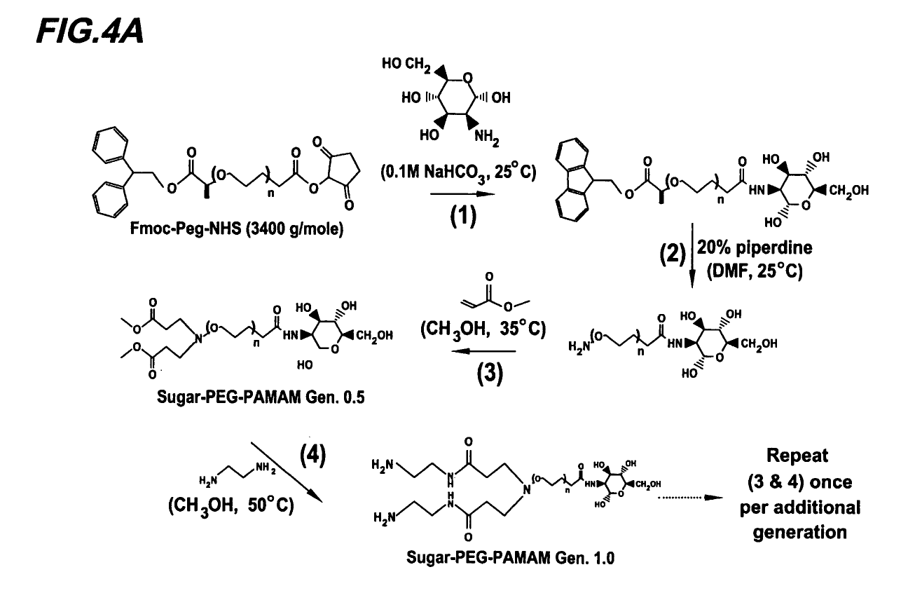

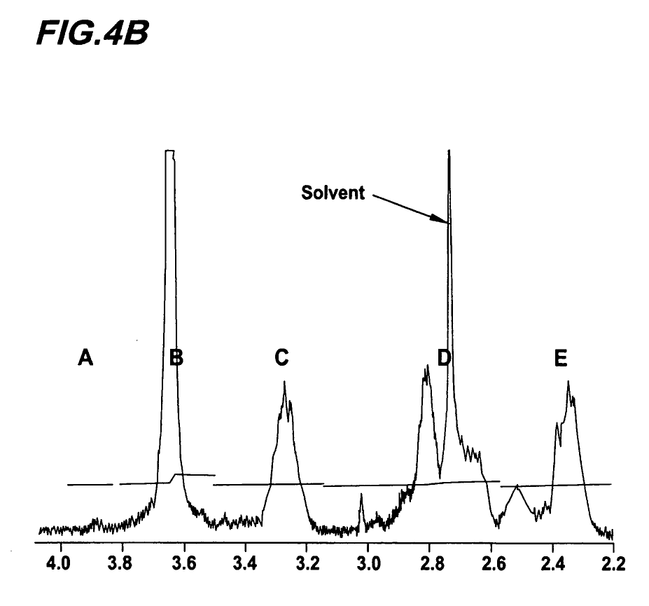

Synthesis: Ligand-functionalized linear-dendritic polymers were synthesized as follows. Fmoc-PEG-NHS (3.5 g; Mₙ=3500, n=72, PDI=1.01) was dissolved in 0.1M NaHCO₃ buffer (0.0375 g/mL) and pH adjusted to pH 8.5 with 1M NaOH. Each of the two sugars were dissolved separately in 0.1M NaHCO₃ buffer (0.03 g/mL), pH adjusted to 8.5, and added to an aliquot of dissolved polymer solution at a molar excess of 10:1 (24 h, 25° C. under N₂ gas). Care was taken to combine sugar and polymer solutions immediately after dissolution of the polymer to avoid premature hydrolysis of the NHS ester. Polymers were recovered by filtration and lyophilization, dissolved in dimethylformamide (DMF, 0.1 g/mL) and added dropwise to a solution of piperidine in DMF to remove the Fmoc protecting group (20% v/v, 30 min, 25° C. under N₂ gas). Following this step, polymers were recovered by precipitation in ice cold diethyl ether and dried overnight under vacuum. Dendrimer synthesis then proceeded by serial Michael addition and amidation steps via addition of methyl acrylate and ethylene diamine, respectively, as described previously (Iyer, et al., Macromolecules, (1998) Volume 31, p 8757, the contents of which are incorporated herein by reference). In general, ligand functionalization and deprotection steps proceeded at 80-85%; all dendrimer synthetic steps proceeded with conversions of 90-100% (FIG. 4). Physical properties of these polymers are listed in Table 1. Qualitatively, the growth of amide (3200-3400 cm⁻¹) and carbonyl (1600-1800 cm⁻¹) peaks during dendrimer synthesis can be seen in FIG. 5. NMR and FTIR peaks for each of the 14 reaction products are listed below. Control polymers (no ligand) were synthesized in parallel with ligand-functionalized species using NH₂—PEG-COOH (Mₙ=3400) as the starting material.

TABLE 1
Theoretical molecular weights and number of amine end groups for ligand-
functionalized PEG-PAMAM hybrid polymers used in this study.

| Polymer | Mₙ (theoretical) | # of Amine End Groups |
|---|---|---|
| Ligand-PEG-PAMAM-G0.0 | 3344 | 1 |
| Ligand-PEG-PAMAM-G1.0 | 3572 | 2 |
| Ligand-PEG-PAMAM-G2.0 | 4028 | 4 |
| Ligand-PEG-PAMAM-G3.0 | 4940 | 8 |
| Ligand-PEG-PAMAM-G4.0 | 6764 | 16 |
| Ligand-PEG-PAMAM-G5.0 | 10412 | 32 |
| Ligand-PEG-PAMAM-G6.0 | 17708 | 64 |

Sugar-Peg-Fmoc. ¹H NMR in CDCl₃: δ_{PEG}(CH₂CH₂O)=3.66 (b); δ_{SUGAR}(CH₂OH)=3.91 (m); δ_{SUGAR}(CHCH₂OH)=3.5 (m); δ_{FMOC}(—CH—)=7.25-7.8 (m). FTIR peaks, v cm⁻¹: 3336, 2885, 1687, 1468, 1344, 1279, 1245, 1111, 964, 845.

Sugar-Peg-NH₂. ¹H NMR in CDCl₃: δ_{PEG}(CH₂CH₂O)=3.62 (b); δ_{SUGAR}(CH₂OH)=3.92 (m); δ_{SUGAR} (CHCH₂OH)=3.51 (m). FTIR peaks, v cm⁻¹: 3347, 2885, 1680, 1470, 1342, 1278, 1109, 964, 843.

Sugar-Peg-G0.5. ¹H NMR in CDCl₃: δ_{PEG} (CH₂CH₂0)=3.61 (b); δ_{SUGAR}(CH₂OH)=3.89 (m); δ_{SUGAR}(CHCH₂OH)=3.5 (m); δ_{PAMAM}(CH₂COOCH₃)=2.48 (m); δ_{PAMAM}(next to tertiary amines)=2.50-2.88 (b); δ_{PAMAM}(CH₂COOCH₃)=3.3 (m). FTIR peaks, v cm⁻¹: 3351, 2885, 1735, 1679, 1467, 1342, 1282, 1112, 965, 844.

Sugar-Peg-G1.0. ¹H NMR in CDCl₃: δ_{PEG}(CH₂CH₂O)=3.65 (b); δ_{SUGAR}(CH₂OH)=3.89 (m); δ_{SUGAR} (CHCH₂OH)=3.52 (m); δ_{PAMAM} (CH₂CONH)=2.43 (m); δ_{PAMAM} (next to primary and tertiary amines)=2.48-2.92 (b); δ_{PAMAM} (CH₂CONHCH₂)=3.33 (m). FTIR peaks, v cm⁻¹: 3336, 2887, 1682, 1471, 1342, 1283, 1111, 962, 843.

Sugar-Peg-G1.5. ¹H NMR in CDCl₃: δ_{PEG} (CH₂CH₂O)=3.64 (b); δ_{SUGAR} (CH₂OH)=3.88 (m); δ_{SUGAR} (CHCH₂OH)=3.5 (m); δ_{PAMAM} (CH₂COOCH₃)=2.45 (m); δ_{PAMAM} (CH₂CONH)=2.15-2.4 (m); δ_{PAMAM} (next to tertiary amines)=2.5-2.88 (b); δ_{PAMAM} (CH₂COOCH₃)=3.6-3.7 (m); δ_{PAMAM} (CH₂CONHCH₂)=3.32 (m). FTIR peaks, v cm⁻¹: 3260, 2880, 1729, 1665, 1550, 1470, 1350, 1260, 1112, 960, 845.

Sugar-Peg-G2.0. ¹H NMR in CDCl₃: δ_{PEG} (CH₂CH₂O)=3.65 (b); δ_{SUGAR} (CH₂OH)=3.89 (m); δ_{SUGAR} (CHCH₂0H)=3.48 (m); δ_{PAMAM} (CH₂CONH)=2.37 (m); δ_{PAMAM} (next to primary and tertiary amines)=2.45-3.0 (b); δ_{PAMAM} (CH₂CONHCH₂)=3.3 (m). FTIR peaks, v cm⁻¹: 3273, 2885, 1653, 1599, 1470, 1360, 1283, 1112, 959, 841.

Sugar-Peg-G2.5. ¹H NMR in CDCl₃: δ_{PEG} (CH₂CH₂O)=3.66 (b); δ_{SUGAR} (CH₂OH)=3.87 (m); δ_{SUGAR} (CHCH₂OH)=3.52 (m); δ_{PAMAM} (CH₂COOCH₃)=2.45 (m); δ_{PAMAM} (CH₂CONH)=2.25-2.42 (m); δ_{PAMAM} (next to tertiary amines)=2.5-2.95 (b); δ_{PAMAM} (CH₂COOCH₃)=3.6-3.7 (m); δ_{PAMAM} (CH₂CONHCH₂)=3.28 (m). FTIR peaks, v cm⁻¹: 3252, 2881, 1736, 1666, 1552, 1467, 1354, 1252, 1113, 957, 843.

Sugar-Peg-G3.0. ¹H NMR in CDCl₃: δ_{PEG} (CH₂CH₂O)=3.67 (b); δ_{SUGAR} (CH₂OH)=3.87 (m); δ_{SUGAR} (CHCH₂OH)=3.54 (m); δ_{PAMAM} (CH₂CONH)=2.38 (m); δ_{PAMAM} (next to primary and tertiary amines)=2.5-3.1 (b); δ_{PAMAM} (CH₂CONHCH₂)=3.3 (m). FTIR peaks, v cm⁻¹: 3253, 3065, 2875, 1662, 1551, 1470, 1354, 1302, 1252, 1107, 955, 853.

Sugar-Peg-G3.5. ¹H NMR in CDCl₃: δ_{PEG} (CH₂CH₂O)=3.65 (b); δ_{SUGAR} (CH₂OH)=3.88 (m); δ_{SUGAR} (CHCH₂OH)=3.55 (m); δ_{PAMAM} (CH₂COOCH3)=2.45 (m); a δ_{PAMAM} (CH₂CONH)=2.15-2.4 (m); δ_{PAMAM} (next to tertiary amines)=2.5-3.0 (b); δ_{PAMAM} (CH₂COOCH₃)=3.6-3.7 (m); δ_{PAMAM} (CH₂CONHCH₂)=3.28 (m). FTIR peaks, v cm⁻¹: 3268, 2870, 1737, 1666, 1552, 1466, 1360, 1256, 1201, 1187, 958, 845.

Sugar-Peg-G4.0. ^{1H} NMR in CDCl₃: δ_{PEG} (CH₂CH₂O)=3.65 (b); δ_{SUGAR} (CH₂OH)=3.88 (m); δ_{SUGAR} (CHCH₂OH)=3.48 (m); δ_{PAMAM} (CH₂CONH)=2.37 (m); δ_{PAMAM} (next to primary and tertiary amines)=2.45-3.1 (b); δ_{PAMAM} (CH₂CONHCH₂)=3.27 (b). FTIR peaks, v cm⁻¹: 3260, 3068, 2881, 1660, 1552, 1470, 1357, 1300, 1260, 1113, 954, 849.

Sugar-Peg-G4.5. ¹H NMR in CDCl₃: δ_{PEG} (CH₂CH₂O)=3.67 (b); δ_{SUGAR} (CH₂OH)=3.92 (m); δ_{SUGAR} (CHCH₂OH)=3.5 (m); δ_{PAMAM} (CH₂COOCH₃)=2.45 (b); δ_{PAMAM} (CH₂CONH)=2.2-2.4 (m); OPAMAM(next to tertiary amines)=2.5-3.2 (b); δ_{PAMAM} (CH₂COOCH₃)=3.6-3.7 (m); δ_{PAMAM} (CH₂CONHCH₂)=3.3 (m). FTIR peaks, v cm⁻¹: 3272, 2873, 1740, 1665, 1556, 1469, 1362, 1260, 1200, 1184, 960, 845.

Sugar-Peg-G5.0. ¹H NMR in CDCl₃: δ_{PEG} (CH₂CH₂O)=3.65 (b); δ_{SUGAR} (CH₂OH)=3.9 (m); δ_{SUGAR} (CHCH₂OH)=3.52 (m); δ_{PAMAM} (CH₂CONH)=2.4 (m); δ_{PAMAM} (next to primary and tertiary amines)=2.5-3.0 (b); δ_{PAMAM} (CH₂CONHCH₂)=3.32 (m). FTIR peaks, v cm⁻¹: 3262, 3073, 2880, 1664, 1555, 1472, 1360, 1301, 1264, 1111, 954, 851.

Sugar-Peg-G5.5. ¹H NMR in CDCl₃: δ_{PEG} (CH₂CH₂O)=3.65 (b); δ_{SUGAR} (CH₂OH)=3.91 (m); δ_{SUGAR} (CHCH₂0H)=3.51 (m); δ_{PAMAM} (CH₂COOCH3)=2.45 (b); δ_{PAMAM} (CH₂CONH)=2.2-2.4 (m); δ_{PAMAM} (next to tertiary amines)=2.5-3.2 (b); δ_{PAMAM} (CH₂COOCH₃)=3.6-3.7 (m); δ_{PAMAM} (CH₂CONHCH₂)=3.3 (m). FTIR peaks, v cm⁻¹: 3274, 2873, 1741, 1670, 1555, 1470, 1362, 1255, 1203, 1190, 960, 847.

Sugar-Peg-G6.0. ¹H NMR in CDCl₃: δ_{PEG} (CH₂CH₂O)=3.66 (b); δ_{SUGAR} (CH₂OH)=3.9 (m); δ_{SUGAR} (CHCH₂OH)=3.55 (m); δ_{PAMAM} (CH₂CONH)=2.4 (m); δ_{PAMAM} (next to primary and tertiary amines)=2.6-3.0 (b); δ_{PAMAM} (CH₂CONHCH₂)=3.2-3.4 (b). FTIR peaks, v cm⁻¹: 3255, 3070, 2884, 1664, 1554, 1472, 1357, 1303, 1256, 1113, 960, 854.

### Example 2

### Characterization of Polymer/DNA Complexes

Gel electrophoresis shift assays: Polymer/DNA complexes (“Polyplexes”) were formed by combining 100 μL of DNA solution (0.1 mg/mL in 25 mM acetate buffer, pH 5.1) to 100 μL of polymer solution (concentration adjusted to reach desired concentration in 25 mM acetate buffer at pH 5.1) in an eppendorf tube and allowing 20 min for complexation. The resultant solutions were diluted in 25 mM acetate buffer and added to gels at a concentration of 100 ng DNA per well (in 20 μL volume) in 10% Ficoll 400 loading buffer (Amersham Pharmacia Biotech, Uppsala, Sweden). Gels were run at 60 V for 1 h using an Embi tec RunOne Electrophoresis Cell (San Diego, Calif.). Bands were visualized by ethidium bromide staining.

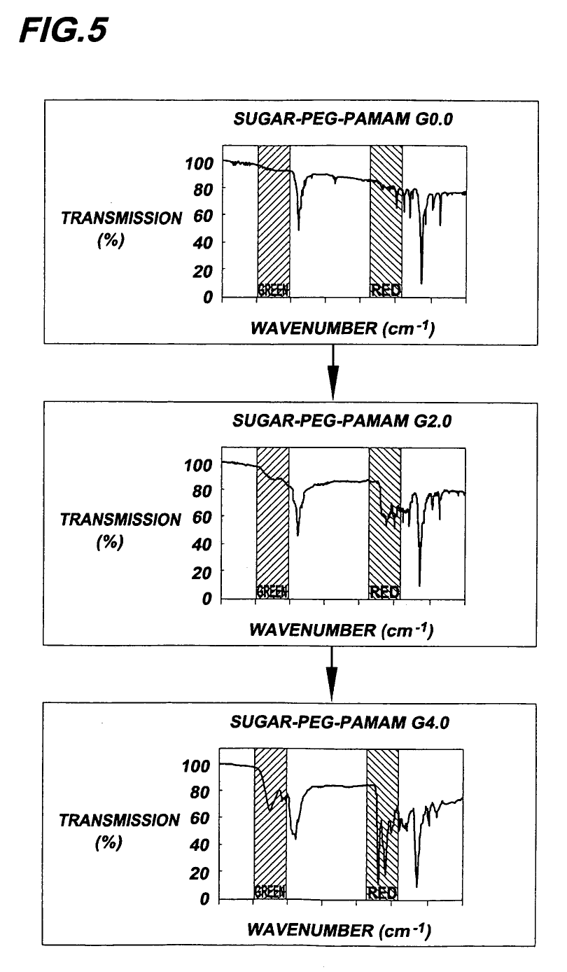

Gel electrophoresis demonstrates binding and charge neutralization of DNA by linear-dendritic polymers incubated at mass ratios of greater than 20:1, 10:1, 5:1, 1:1, and 1:1 for generations 2.0, 3.0, 4.0, 5.0, and 6.0, respectively (FIG. 6A). The nature of this trend is consistent with intuition, as the exponentially increasing number of amines with increasing dendrimer generation results in higher charge density with increasing dendrimer size.

Dynamic light scattering (DLS): Dynamic light scattering (DLS) measurements were used to measure the size of polymer-DNA complexes. Complexes were prepared as described above. Correlation functions were collected at a scattering angle of 90°, and the sizes of particles were determined using the MAS option of the company's particle sizing software package (version 2.30) assuming the refractive index and viscosity of pure water at room temperature. Particle sizes, obtained in triplicate, are given as effective diameters assuming a log-normal distribution.

Dynamic light scattering (DLS) suggests that polyplexes of generations 3.0, 4.0, and 6.0 average around 150 nm in diameter, under the reported cutoff of around 200 nm required for efficient cellular uptake (FIG. 6B).¹² Generation 5.0 polyplexes form larger particles with DNA, a seemingly anomalous result that was nevertheless highly repeatable. The large size of G2.0 polyplexes reflects the fact that little DNA binding and charge neutralization occurred in these systems. In all cases, mass ratios ranging from 0.1 to 200 were tested and polyplex size was shown to be relatively insensitive to mass ratio above the ratio at which complexation occurs in each system, suggesting that in all cases polyplexes consist of a single DNA plasmid and that excess polymers remain dispersed in solution. Thus, the particle diameters listed in FIG. 6B represents average diameters for an evenly weighted range of mass ratios up to 200.

Transmission electron microscopy (TEM): Transmission electron micrographs were obtained using a JEOL 2000FX operating at 200 kV. TEM samples were prepared on 400-mesh, Formivar carbon-coated copper TEM grids by first depositing a small aliquot of the above complex solution (5 μL) onto the grid and allowing 15 minutes for evaporation of the solvent. A small drop (30 μL) of staining solution containing 0.5% RuO₄ was then placed on the sample and allowed to evaporate for 1 h prior to imaging.

TEM of G6.0 polyplexes shows narrowly dispersed, roughly spherical complexes with an outer corona of approximately 6-8 nm, consistent with the expected size of PEG-PAMAM G6.0 (FIG. 6C).¹³ In all of the above cases, complexes were formed prior to assay by incubating dilute solutions of plasmid DNA encoding firefly luciferase (6.2 kb, 2.05×10⁶ g/mol, 0.1 mg/mL, 25 mM acetate buffer, pH 5.1) with equal volumes of solutions containing polymers (in 25 mM acetate buffer, pH 5.1 at appropriate concentrations to achieve the indicated mass ratios) for 20 min at room temperature.

### Example 3

To evaluate the ability of polyplexes to transfect target cells via receptor-mediated uptake, we transfected two cell types, P388D1 murine macrophages bearing the mannose receptor and HepG2 human hepatocytes bearing the asialoglycoprotein receptor (for galactosylated ligands).¹⁵⁻¹⁸ All transfection assays were performed in quadruplicate in accordance with the following protocol. All materials, buffers, and media were sterilized prior to use. HepG2 cells grown in 96-well plates at an initial seeding density of 5000 cells/well in 150 μL/well of growth medium (90% Dulbecco's modified Eagle's medium supplemented with 10% fetal bovine serum, 100 units/mL penicillin, and 100 μg/mL streptomycin). P388D1 cells were grown in separate 96-well plates at an initial seeding density of 50000 cells/well in 150 μL/well of growth medium (90% RPMI 1640 medium supplemented with 10% fetal bovine serum, 100 units/mL penicillin, 100 μg/mL streptomycin, 2.5 mg/mL D-glucose, 10 mM HEPES, and 1 mM sodium pyruvate). Cells were allowed to attach and proliferate for 24 h in an incubator.

Polymers were dissolved in sterile 25 mM acetate buffer (concentrations ranging from 2-12 mg/mL) and arrayed into a 96-well plate (25 μL/well total polymer solution with concentrations adjusted as appropriate to yield polymer/DNA ratios ranging from 10:1 to 200:1). Polymer/DNA complexes were formed by the addition of 25 μL/well of 0.06 mg/mL pCMV-Luc in 25 mM acetate buffer. Polymer and DNA solutions were vigorously mixed using a multichannel pipettor upon addition of DNA solutions and subsequently incubated for 20 min to allow for complexation. Thirty μL/well aliquots of the above complex solutions were then transferred into each well of a 96-well plate containing 200 μL/well of either serum-free Opti-MEM medium (Invitrogen Corporation, Carlsbad, Calif.) or 10% serum-containing growth medium. Growth medium was removed from cells and 150 μL/well of complex-plus-medium solution was added. Controls employing PEI were prepared exactly as above to yield polymer DNA ratios ranging from 0.5:1 to 10:1, and in all cases optimized formulations are reported as positive controls. Naked DNA and no-DNA controls were also prepared as above, and each 96-well plate included appropriate positive and negative controls as internal standards. In all cases, wells contained 587 ng DNA/well at indicated polymer/DNA ratios.

Following incubation of complex-containing medium solutions with cells for 4 h, solutions were removed and replaced with 10% serum-containing growth medium. Cells were incubated for an additional 72 h, and luciferase expression was determined using the commercially available Bright-Glo® luciferase assay kit (Promega, Madison, Wis.). Luminescence was quantified in solid, flat-bottom, white polypropylene 96-well plates using a bioluminescent plate reader. Luminescence was expressed in relative light units and was not normalized to total cellular protein in this assay.

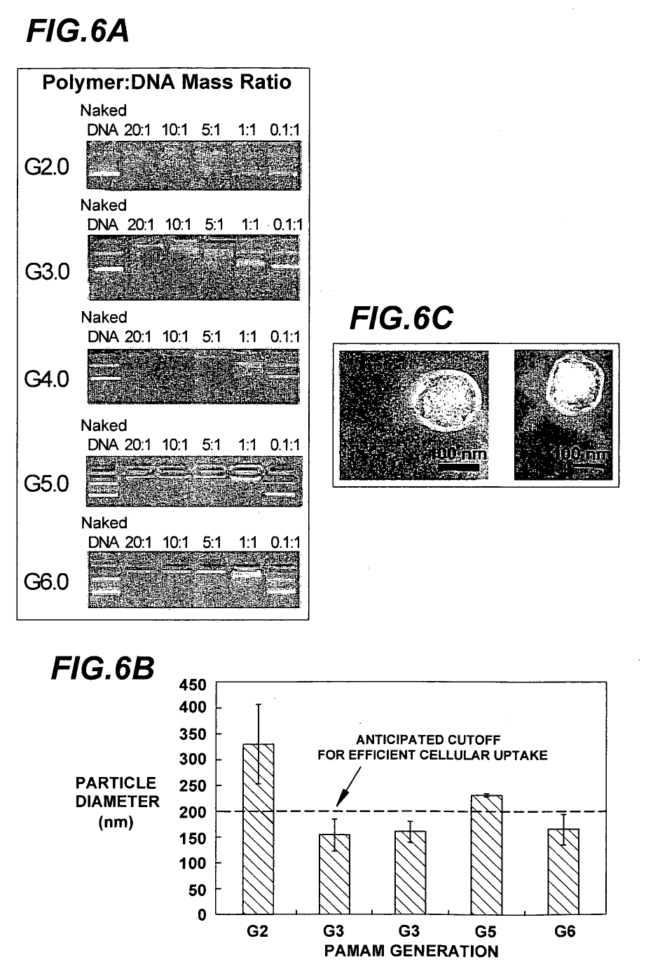

FIG. 7A shows levels of luciferase reporter gene expression in macrophages (in the absence of serum) with optimized formulations of ligand-functionalized polyplexes, control polyplexes bearing no ligand, ligand-functionalized polyplexes in the presence of excess soluble ligand, and PEI. Generation 6.0, mannose-bearing polyplexes demonstrate transfection 1.6- to 1.8-fold higher than PEI, the most efficient commercially available polymer for in vitro transfections. Generation 5.0 polyplexes mediate reporter expression levels approximately 1.3-fold higher than PEI, while G4.0 polyplexes (as well as G3.0 and G2.0, data not shown) transfect at low levels comparable to naked DNA. The highest transfection levels were observed in polymer/DNA ratios under 50 in all systems (under 20 in G6.0), presumably owing to the effects of toxicity at high concentrations. Polyplexes with no mannose ligand exhibited significantly lower transfection efficiencies, and competitive inhibition of mannose receptors by an excess of soluble ligand virtually silenced reporter gene expression without affecting expression levels in positive and negative controls (FIG. 7A). These data further support the hypothesis of cellular internalization by means of specific, receptor-mediated endocytosis. Finally, to probe the serum stability of PEGylated polyplexes, macrophages were transfected in the presence of 10% serum containing medium (FIG. 7B). Four-fold transfection enhancements were observed relative to PEI, most likely owing to the “stealth” effect imparted by PEG, which is known to reduce particle agglomeration by attenuating opsonization of serum proteins.¹⁹

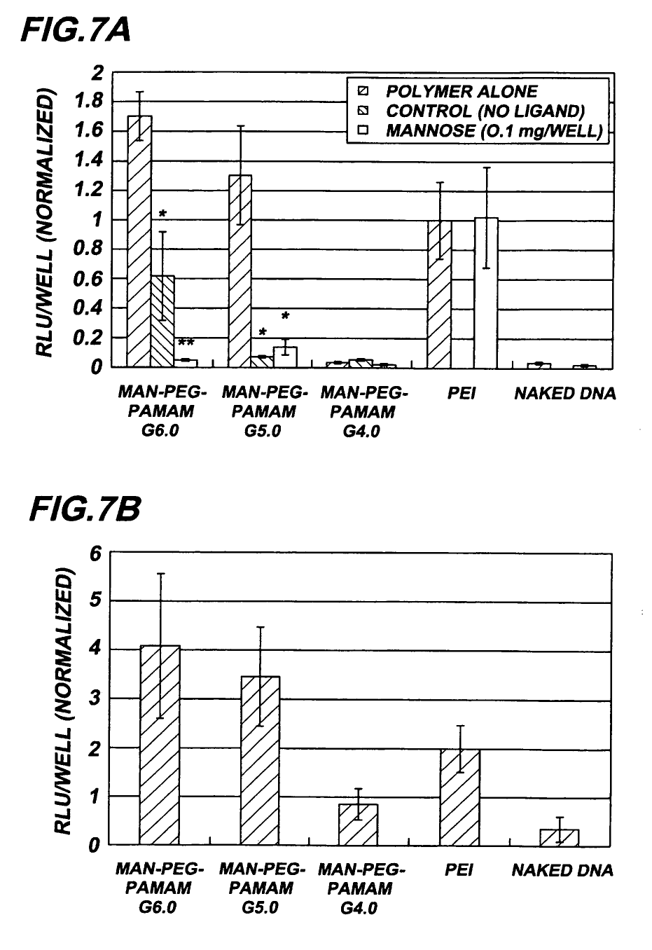

Transfection of HepG2 hepatocytes by linear-dendritic polyplexes bearing the galactose ligand is shown in FIG. 8. In the absence of serum, optimized formulations of generation 6.0 and 4.0, ligandfunctionalized polyplexes transfect significantly more efficiently (p<0.06) than control polymers with no ligand (FIG. 8A). Moreover, generations 6.0, 5.0, and 4.0 targeted systems mediate transfection levels within one order of magnitude of PEI in the absence of serum, and as much as eight-fold higher than PEI in the presence of serum (FIGS. 8A and 8B). Optimal polymer/DNA mass ratios were in the range of 100-200 for all systems studied. Taken together, these data suggest that hepatocytetargeted polyplexes are serum stable and demonstrate enhanced transfection owing to a cell-specific, receptor-mediated process. Interestingly, expression levels were unaffected by the presence of an excess of soluble galactose, a finding that may owe to the multivalent nature of ligand binding by the asialoglycoprotein receptor, suggesting that multivalent ligand presentation via synthetic, multimeric galactose ligands may yield enhanced targeting relative to monomeric species.^{20,21}

### Example 4

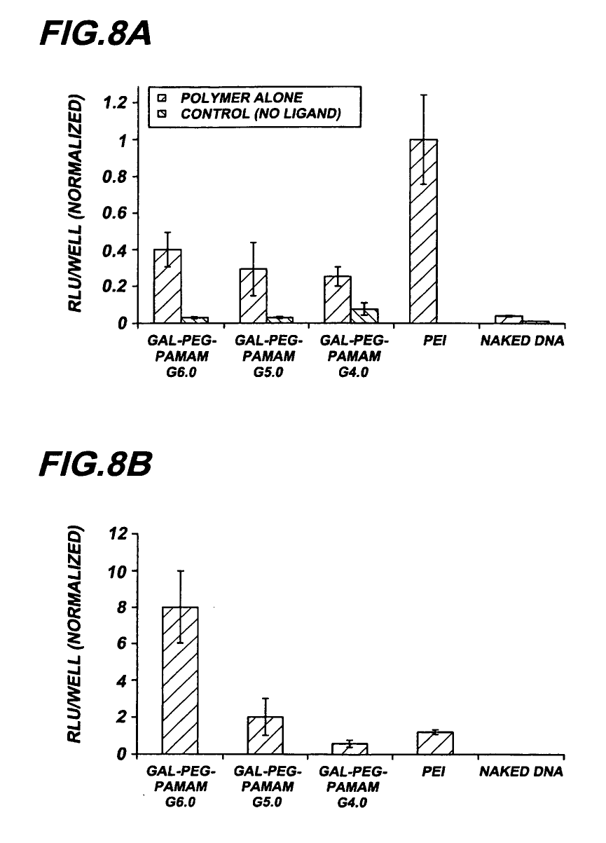

To assess the cellular toxicity of linear-dendritic hybrid polymer-based systems, an MTT assay was performed to measure the relative viability of cells treated with varying polymer/DNA mass ratios. A range of polymer/DNA mass ratios were studied, corresponding to concentrations equal to and above those at which optimal transfection levels were observed. Cells were seeded in clear flat-bottom 96-well plates and transfected exactly as previously described. After 72 h, cell metabolic activity was assayed using the MTT cell proliferation assay kit (ATCC, Manassas, Va.). Initially, a 10 μL aliquot of MTT assay reagent was added to each well. After incubating for two hours, 100 μL of detergent reagent was added. The plate was then covered and left in the dark for 4 h, after which optical absorbance was measured at 570 nm using a microplate absorbance reader. Background (media plus MTT assay reagent plus detergent reagent with no cells present) was subtracted from the value of each well, and all values were normalized to the value of control (untreated) cells. In P388D1 macrophages (FIG. 9A), cells which we have found to be highly sensitive to environmental conditions in culture, no measurable toxicity was observed in G4.0-based systems over the entire concentration range studied. More significant toxicity was observed at high mass ratios in G5.0 (60-80% viability relative to untreated controls) and G6.0 systems (50-70%), though these toxicities were primarily observed at concentrations higher than those optimal for transfection. In HepG2 hepatocytes (FIG. 9B), no measurable toxicity was observed in G4.0 and G5.0 systems at polymer/DNA mass ratios up to 200; in G6.0, moderate toxicity became apparent at ratios of 150 and above. In all cases, linear-dendritic systems failed to display toxicity until concentrations reached one to two orders of magnitude greater than those at which PEI was toxic.

### Example 5

Recently, several investigators have demonstrated that short peptides have the ability to direct the selective uptake of bacteriophage, adenovirus, and nanoparticulate systems into desired organs or tissues. We synthesized hybrid polymer systems capable of delivering genes selectively to tumors in vivo by targeting internalizable surface antigens on tumor or tumor endothelial cells by functionalizing polymer systems with “homing peptides.” The first peptide chosen is an eight-amino acid peptide (sequence: WIFPWIQL) that selectively binds glucose-responsive protein 78 kDa, a transmembrane stress response protein that is selectively expressed on the surface of a number of human breast and prostate cancers.

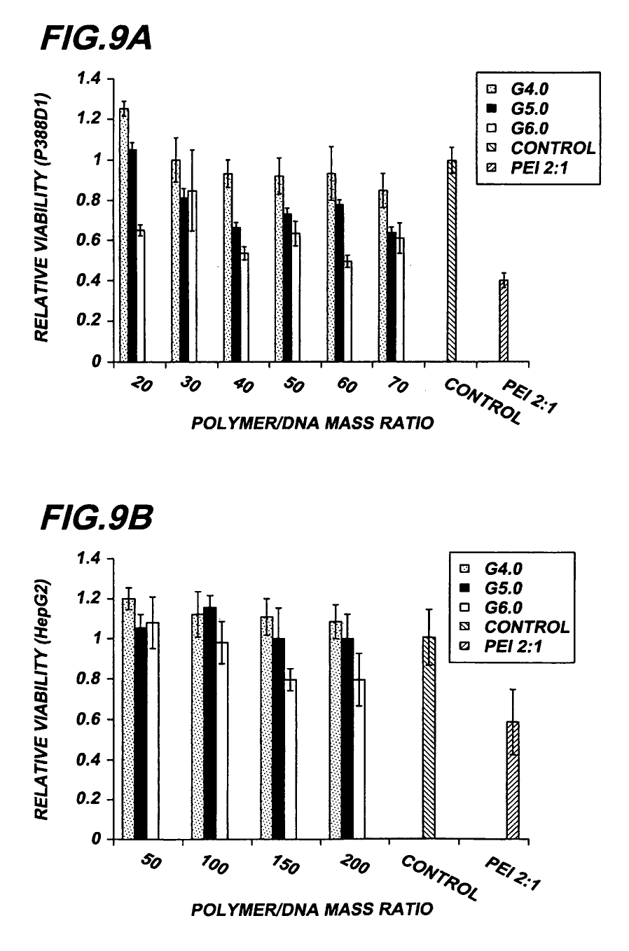

In order to synthesize hybrid polymers functionalized with targeting peptides, we had to first redesign the synthetic procedure to allow for all-aqueous processing conditions that would be favorable to biological entities like peptides that are sensitive to degradation and denaturation by heat or organic solvents. An exemplary three-step synthetic protocol exploits the commercial availability of disulfide-core PAMAM dendrimers (Dendritic Nanotechnologies, Midland, Mich.) and the selective reactivity of maleimide functional groups with thiols versus amines (FIG. 10). First, disulfide-core PAMAM dendrimers (Dendritic Nanotechnologies, Midland, Mich.) were dissolved in 1×TAE buffer with a 10-fold molar excess of dithiothreitol (DTT, a reducing agent) and stirred for 48-72 h under nitrogen gas at room temperature. Next, the solution was dialyzed (MWCO 1000) against pure water to remove all buffer and excess DTT. In a separate vial, NHS ester-Peg-Maleimide (Nektar Therapeutics, Birmingham, Ala.) was dissolved in 1×PBS buffer and added to an equal volume of targeting peptide (sequence: N terminus-WIFPWIQL-C terminus) dissolved in pure DMSO and stirred at room temperature under nitrogen gas for 60-90 minutes. Next, the reduced, dialyzed PAMAM dendrimer solution was adjusted to 1×PBS (using 10×PBS stock solution) and added to the Peg-Peptide reaction mixture, which was stirred for an additional two days under nitrogen gas at room temperature. Concentrations of all reactant solutions were adjusted to yield a 1:1:1 molar ratio of Peptide:Peg:PAMAM. After 48 h, the solution was dialyzed (MWCO 3500) against pure water to remove DMSO, unreacted peptides, and buffers, and then stored at 4° C. Analytical analysis of reactants and products was performed using NMR, FTIR, and Ellman's reagent (a conventional technique for quantifying thiol concentrations in solution).

### Example 6

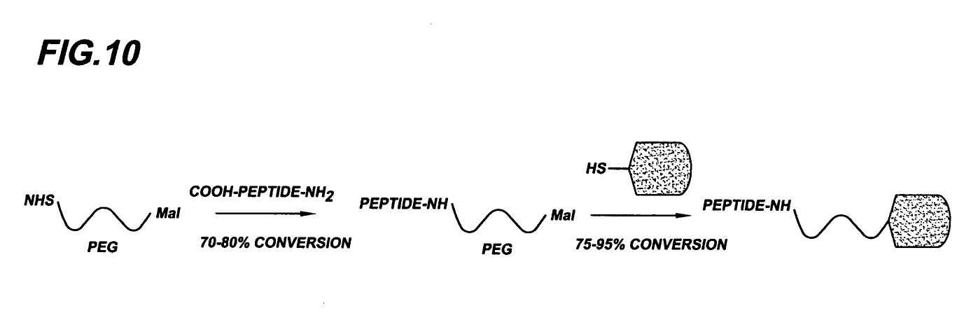

Following the synthesis of peptide-functionalized polymers, we probed the DNA binding and transfection properties of these systems by following the procedures outlined above for sugar-functionalized systems. We found (Table 2) that particles are small (140-180 nm) and that Generation 5.0 systems can transfect DU145 human prostate cancer cells expressing the GRP78 receptor at levels that are 10-fold higher than an optimized formulation of PEI and significantly more efficiently than polymers lacking the targeting ligand or in the presence of a competing antibody (50:1 dilution of anti-GRP78 polyclonal antisera; see FIG. 11).

TABLE 2
Particle Sizes

| Complex | Diameter (nm) |
|---|---|
| G5.0-pLuc | 172 +/− 3 |
| G6.0-pLuc | 137 +/− 2 |
| G7.0-pLuc | 146 +/− 5 |

### Example 7

In order to characterize tumor-specific transfection by hybrid polymer systems in vivo, we implant DU145-tumors into immunodeficient nude mice. Tumor cells (10⁶ cells/mouse) are implanted subcutaneously and allowed to reach a size of 50 mm³. Mice are injected with polymer-DNA complexes containing either peptide-modified or unmodified (control) polymers via intravenous or intratumoral routes. After 3, 5, and 7 days, luciferase gene expression is imaged using a Xenogen IVIS200 Imaging System. Following successful transfection, identical mice are transfected with a plasmid encoding the HSVtk gene (Herpes simplex virus thymidine kinase), an enzyme which, when administered with gancyclovir promotes apoptosis of transfected cells by inhibiting DNA polymerase. Efficacy in this later study is measured by decrease in tumor size, selective destruction of tumor tissue as measured by histopathology, and increased duration of animal survival.

### Example 8

DNA caccination is the process of gene delivery to antigen-presenting cells (APCs) for the purpose of stimulating a protective immune response. DNA vaccination makes possible the stimulation of both humoral and cellular branches of the immune system. Further, this approach offers the possibility of providing protective immunity without the necessity of exposing patients to a potentially infectious agent such as an attenuated virus or bacterium. Additionally, because DNA vaccines can potentially be produced and scaled rapidly to meet the demands of any particular application, and because the requirements for delivery and formulation of DNA vaccines are fairly insensitive to the particular antigen being encoded, this technology may offer valuable solutions in cases where large amounts of vaccines are needed rapidly or when antigens are highly dangerous or potent, thus making traditional vaccination strategies unusable (i.e., HIV).

Many APCs (e.g., dendritic cells, among the more potent APCs in the body) express mannose-specific pattern recognition receptors. As a result, targeting the mannose receptor using gene delivery vectors can be a useful method for transfection of APCs. The mannose-functionalized hybrid polymers described above were used to transfect APCs in vivo. A series of protocols were developed for investigating transfection of dendritic cells, subsequent antigen presentation, and the stimulation of humoral (antibody-mediated) responses in a series of mouse models.

Antibody Production

To measure antibody production in vivo, the following procedure was used. Mice (C57BL/6 male mice, 6-8 weeks old) received a single 200 μL injection containing 100 μg DNA (encoding the beta-galactosidase protein driven by a CMV promoter) formulated in a 10:1 (m:m) ratio of polymer:DNA in 5% glucose intradermally. Negative control mice received saline only. All mice received prime injections on day 0, boost injections (identical to primes) on day 28, and were challenged on day 56 with 50 μg of soluble protein antigen (beta-galactosidase). Blood samples were drawn from the tail vein of all mice every 7 days beginning on day 7 and ending on day 84. Anti-beta-galactosidase antibody titers in serum samples were determined by a conventional ELISA assay.

Dendritic Cell Transfection In Vivo

To measure the transfection of dendritic cells in vivo, complexes containing DNA encoding the thy 1.1 transmembrane protein driven by a CMV promoter were formulated exactly as above and injected into C57BL/6 male mice intradermally. After 48 h, mice were sacrificed by CO₂ asphyxiation and spleens and lymph nodes were processed into single cell suspensions and labeled with antibodies to CD11c (dendritic cell marker) and thy1.1. Thy1.1 expression levels on CD11c+ cells were evaluated and compared with negative controls (saline only, naked DNA, and identical polymer-DNA complexes containing blank plasmid).

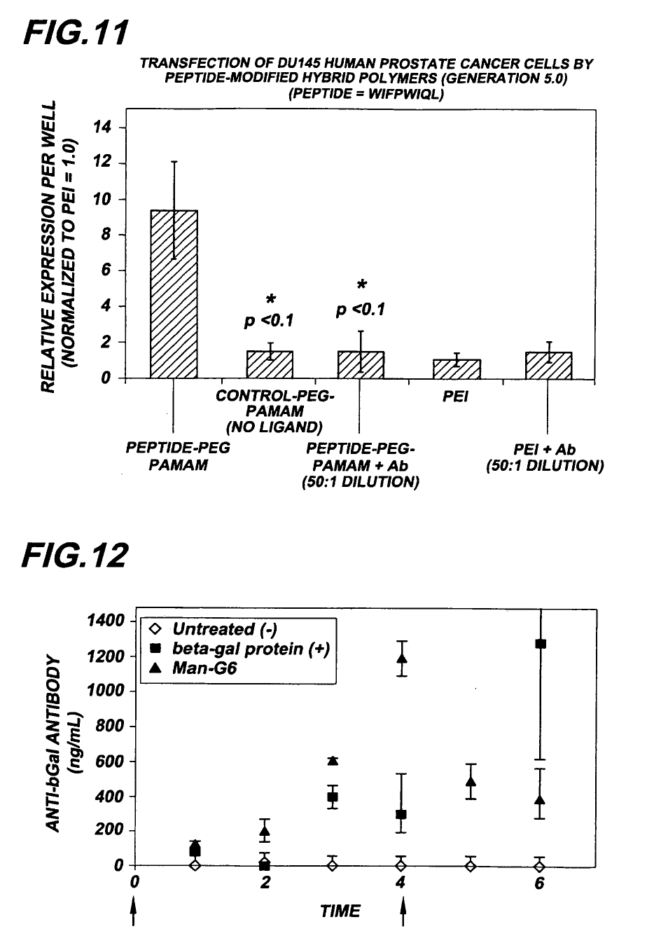

In vivo antibody responses are shown below. FIG. 12 shows antibody levels in representative mice that were untreated or treated with soluble beta-galactosidase protein (a model antigen used as a positive control) or linear-dendritic complexes encoding the beta-galactosidase protein. Arrows indicate the dates of prime (week zero) and boost (week four) injections. Table 3 shows the maximum serum dilution at which anti-beta-galactosidase antibodies could still be detected in a cohort of mice treated as described.

TABLE 3
Maximum serum dilution at which anti-beta-galactosidase antibodies
can still be detected

| | Untreated | | | | | | | |
|---|---|---|---|---|---|---|---|---|
| Week # | (−) | βgal (+) | βgal (+) | ManG6 | ManG6 | ManG6 | ManG6 | ManG6 |
| 1 | — | 20 | 100 | 20 | 100 | 100 | 100 | 100 |
| 2 | 20 | — | — | 20 | — | — | 100 | 20 |
| 3 | — | 20 | 500 | — | 20 | 20 | 2500 | 20 |
| 4 | — | 100 | 2500 | 20 | 20 | 100 | 12500 | 100 |
| 5 | — | — | 2500 | — | — | 20 | 100 | — |
| 6 | — | 500 | 2500 | — | — | — | 500 | — |

T Cell Activation In Vivo

To measure T cell activation in vivo, we used the following protocol. First, 2C T-cells were freshly isolated from the lymph nodes and spleen of 2C TCR transgenic mice. Two million naive 2C T cells were injected intravenously into C57BL/6 mice (<6-8 weeks old, Jackson Lab). At the same time, polymer-DNA complexes prepared as described above (containing the plasmid pCIneohsp65-p1) were injected intradermally into the abdomen of the same recipients. Seven days later, the mice were sacrificed and their spleens were harvested and processed into single-cell suspensions. CD8+ 2C T cells were assayed by immunostaining of an early activation marker CD69 followed by fluorescence activated cell sorting (FACS). CD69 levels in hybrid polymer-treated mice were compared with those in mice treated with saline or naked DNA only (negative controls). Clonal expansion of CD8+ cytotoxic T cells in vivo was observed (data not shown).

### REFERENCES

[1] I. M. Verma, N. Somia, Nature 1997, 389, 239.
[2] I. M. Verma, M. D. Weitzman, Annu. Rev. Biochem. 2005, 74, 711.
[3] E. Wagner, Pharm. Res. 2004, 21, 8.
[4] E. Neumann, M. Schaefer-Ridder, Y. Wang, P. Hofschneider, EMBO J. 1982, 1, 841.
[5] S. Mehier-Humbert, R. H. Guy, Adv. Drug Delivery Rev. 2005, 57, 733.
[6] D. Luo, W. Saltzman, Nat. Biotechnol. 2000, 18, 33.
[7] D. J. Glover, H. J. Lipps, D. A. Jans, Nat. Rev. Genet. 2005, 6, 299.
[8] F. M. Munkonge, D. A. Dean, E. Hillery, U. Griesenbach, E. W. Alton, Adv. Drug Delivery Rev. 2003, 55, 749.
[9] D. V. Schaffer, N. A. Fidelman, N. Dan, D. A. Lauffenburger, Biotechnol. Bioeng. 2000, 67, 598.
[10] N. D. Sonawane, F. C. Szoka, A. S. Verkman, J. Biol. Chem. 2003, 278, 44826.
[11] J. Iyer, K. Fleming, P. T. Hammond, Macromolecules 1998, 31, 8757.
[12] J. Rejman, V. Oberle, I. S. Zuhorn, D. Hoekstra, Biochem. J. 2004, 377, 159.
[13] D. A. Tomalia, Prog. Polym. Sci. 2005, 30, 294.
[15] S. S. Diebold, C. Plank, M. Cotten, E. Wagner, M. Zenke, In Synthetic DNA delivery systems (Eds.: D. Luo, W. M. Saltzman), Kluwer, New York 2003.
[16] B. Hoflack, S. Kornfeld, Proc. Natl. Acad. Sci. U.S.A. 1985, 82, 4428.
[17] J. S. Remy, A. Kichler, V. Mordvinov, F. Schuber, J. P. Behr, Proc. Natl. Acad. Sci. U.S.A. 1995, 92, 1744.
[18] R. J. Stockert, Physiol. Rev. 1995, 75, 591.
[19] J. M. Harris, R. B. Chess, Nat. Rev. Drug Discovery 2003, 2, 214.
[20] D. T. Connolly, R. R. Townsend, K. Kawaguchi, W. R. Bell, Y. C. Lee, J. Biol. Chem. 1982, 257, 939.
[21] Y. C. Lee, R. T. Lee, Acc. Chem. Res. 1995, 28, 321.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A linear-dendritic hybrid polymer for encapsulating biologically active materials, comprising:
a ligand for a predetermined target;
a dendron; and
a polyethylene glycol (PEG) chain linking the ligand to the dendron.

2. The hybrid polymer of claim 1, wherein the PEG chain is branched, and wherein the hybrid polymer comprises more than one ligand linked to the dendron by the PEG chain.

3. The hybrid polymer of claim 1, wherein the predetermined target is multivalent.

4. The hybrid polymer of claim 1, wherein the PEG chain comprises at least 9 repeat units.

5. The hybrid polymer of claim 1, wherein the PEG chain comprises at least 100 repeat units.

6. The hybrid polymer of claim 1, wherein the PEG chain comprises at least 500 repeat units.

7. The hybrid polymer of claim 1, wherein the PEG chain comprises at least 1000 repeat units.

8. The hybrid polymer of claim 1, wherein the PEG chain comprises at least 5000 repeat units.

9. The hybrid polymer of claim 1, wherein the PEG chain comprises at least 10,000 repeat units.

10. The hybrid polymer of claim 1, wherein the dendron is a G3-G10 dendron.

11. The hybrid polymer of claim 10, wherein the dendron is a G4-G6 dendron.

12. The hybrid polymer of claim 1, wherein the dendron comprises poly(amidoamine), polylysine, or polypropylenimine, has a peptide-based dendrimer composition, has a nucleic acid based composition, or has a degradable cationic dendrimer composition.

13. The hybrid polymer of claim 1, wherein the dendron comprises functional groups having a pKa between about 5.5 and about 7.5.

14. The hybrid polymer of claim 1, wherein the ligand comprises a nucleic acid ligand, oligonucleotide, oligopeptide, polysaccharide, low-density lipoprotein (LDLs), folate, transferrin, asialycoprotein, gp120 envelope protein of the human immunodeficiency virus (HIV), enzymatic receptor ligand, sialic acid, glycoprotein, lipid, small molecule, bioactive agent, biomolecule, immunoreactive fragments such as the Fab, Fab′, or F(ab′)₂ fragments, protein, lipid, small molecule, bioactive agent, biomolecule, antibody, or antibody fragment:

15. The hybrid polymer of claim 14, wherein the ligand is retained on the PEG chain through covalent or non-covalent interactions.

16. The hybrid polymer of claim 15, wherein the non-covalent interactions are selected from host-guest interactions, hydrogen bonding, metal coordination, hydrophobic interactions, or the interaction between biotin and avidin or streptavidin.

17. The hybrid polymer of claim 1, wherein free ends of the dendron comprise one or more of biotin, streptavidin, avidin, nitrile, amide, ester, thiol, halogen, tosylate, hydroxyl, alkyl, aryl, and alkylaryl.

18. A nanoparticle for use in encapsulating a biologically active agent, comprising:
a quantity of the biologically active agent surrounded by a shell comprising the hybrid polymer of claim 1.

19. The nanoparticle of claim 18, wherein at least a portion of the biologically active agent interacts with free ends of the dendron via a non-covalent interaction.

20. The nanoparticle of claim 19, wherein the non-covalent interaction is selected from host-guest interactions, hydrogen bonding, metal coordination, hydrophobic interactions, pi-bonding, charge interactions, and the interaction between biotin and avidin or streptavidin.

21. The nanoparticle of claim 18, wherein the nanoparticle is between 25 nm and 2 micron in diameter.

22. The nanoparticle of claim 21, wherein the nanoparticle is between 25 nm and 100 nm in diameter.

23. The nanoparticle of claim 21, wherein the nanoparticle is between 100 nm and 500 nm in diameter.

24. The nanoparticle of claim 21, wherein the nanoparticle is between 500 nm and 1 micron in diameter.

25. The nanoparticle of claim 21, wherein the nanoparticle is between 1 and 2 microns in diameter.

26. The nanoparticle of claim 18, wherein the biologically active agent is a polynucleotide, a small molecule, a bioactive agent, a polypeptide, a growth factor, or a glycosaminoglycan.

27. A composition for delivering a biologically active agent to a patient, comprising:
a plurality of nanoparticles according to claim 18.

28. The composition of claim 27, further comprising a carrier.

29. The composition of claim 27, wherein the composition is suitable for administration by injection, as a suppository, orally, as an inhalant, or topically.

30. A method of producing a ligand-functionalized polyethylene glycol (PEG)-dendrimer hybrid polymer, comprising:
attaching a predetermined ligand to a free end of a PEG chain using a covalent or non-covalent interaction; and
using a second free end of the PEG chain as the core of a dendron.

31. The method of claim 30, wherein using a second free end comprises alternately reacting a primary amine in chemical communication with the PEG chain with methyl acrylate and ethylene diamine.

32. The method of claim 30, wherein the PEG chain has more than two free ends, and wherein attaching comprises attaching a ligand to all but one of the free ends.

33. The method of claim 30, wherein the non-covalent interactions is selected from host-guest interactions, hydrogen bonding, metal coordination, hydrophobic interactions, and the interaction between biotin and avidin or streptavidin.

34. The method of claim 30, wherein using a second free end comprises synthesizing a G3-G10 dendron.

35. The method of claim 30, wherein using a second free end comprises synthesizing a G4-G6 dendron.

36. The method of claim 30, further comprising modifying at least a portion of free ends of the dendron.

37. The method of claim 36, wherein the portion is modified to include negatively charged groups, biotin, avidin, streptavidin, nitrile, amide, ester, thiol, halogen, tosylate, hydroxyl, alkyl, aryl, and alkylaryl.

38. A method of encapsulating a biologically active material, comprising:
providing a hybrid polymer comprising a ligand for a predetermined target, a dendron, and a PEG chain linking the ligand to the dendron; and
incubating the hybrid polymer with the biologically active material under conditions where the hybrid polymer forms vesicles surrounding a quantity of the biologically active material.

39. The method of claim 38, wherein the biologically active material is a polynucleotide, a small molecule, a bioactive molecule, a polypeptide, a growth factor, or a glycosaminoglycan.

40. The method of claim 38, wherein at least a portion of the biologically active material interacts with free ends of the dendron via a non-covalent interaction.

41. The method of claim 38, wherein the biologically active material interacts with the dendron through electrostatic interactions, host-guest interactions, hydrogen bonding, metal coordination, hydrophobic interactions, pi-bonding, charge interactions, or the interaction between biotin and avidin or streptavidin.

42. The method of claim 38, wherein at least a portion of the vesicles are between 25 nm and 2 micron in diameter.

43. The method of claim 42, wherein at least a portion of the vesicles are between 25 nm and 100 nm in diameter.

44. The method of claim 42, wherein at least a portion of the vesicles are between 100 nm and 500 nm in diameter.

45. The method of claim 42, wherein at least a portion of the vesicles are between 500 nm and 1 micron in diameter.

46. The method of claim 42, wherein at least a portion of the vesicles are between 1 and 2 microns in diameter.
